(19) United States
(12) Patent Application Publication
(10) Pub. No.: US 2017/0020958 A1
(43) Pub. Date: Jan. 26, 2017

(54) STEM CELLS FOR ANTI-ANGIOGENIC THERAPY IN AGE-RELATED MACULAR DEGENERATION, DIABETIC RETINOPATHY, CORNEAL VASCULARISATION AND CANCER

(71) Applicant: CRYOCORD SDN. BHD., Cyberjaya (MY)

(72) Inventor: Then Khong YONG, Kuala Lumpur (MY)

(21) Appl. No.: 15/098,185

(22) Filed: Apr. 13, 2016

(30) Foreign Application Priority Data
Jul. 23, 2015 (MY) PI 2015702405

(51) Int. Cl. A61K 38/17; C12N 5/0775; A61K 38/45; A61K 35/28; C12N 9/12

(52) U.S. Cl. CPC A61K 38/179; A61K 35/28; C12N 9/1205; C12Y 207/01112; A61K 38/45; C12N 5/0668; C12N 2510/00

(57) ABSTRACT

The present invention relates to production of a stem cell expressing an anti-angiogenic protein. The stem cells are used to inhibit angiogenesis for treatment of macular degeneration, corneal vascularisation, cancer and diabetic retinopathy.

### FIELD OF INVENTION

The present invention relates to a method for long term generation of vascular endothelial growth factor receptors (VEGFRs) with use of mesenchymal stem cells isolated from Wharton's jelly of umbilical cord (WJ-MSCs). The WJ-MSCs are to be used for inhibiting angiogenesis in treatment of diseases related to uncontrollable growth of blood vessels, in particular macular degeneration, diabetic retinopathy, corneal vascularisation and cancer. The present invention is particularly relevant in field of cell therapy.

### BACKGROUND OF INVENTION

Angiogenesis is defined as growth of new blood vessels in body which branch out from existing vasculature. Beginning in utero, angiogenesis occurs throughout human life. Blood vessels are vital and needed in all tissues for diffusion exchange of nutrients and metabolites. Human body controls angiogenesis by maintaining the balance of growth and inhibitory factors. Angiogenesis is controlled by a number of growth and inhibitory factors. Angiogenin, angiopoietin-1, interleukin-8 and placental growth factor are some of the growth factors while, the inhibitory factors include angioarrestin, chondromodulin, heparinases, interleukin-12 and troponin I.

However, upsetting the balance between growth and inhibitory factors will cause abnormal growth of blood vessels which, in turn causes many diseases including macular degeneration, cancer, diabetic retinopathy, lymphangiogenesis and retinal neovascularisation.

Anti-angiogenic treatments are a point of growing interest as angiogenesis is known to be main factor for spread of cancers with excessive growth of blood vessels. Also, abnormal blood vessels develop under macula and break, bleed, and leak fluid, causing macular degeneration in older people. Anti-angiogenic agents have been widely used for inhibiting growth of blood vessels. They can be primarily classified into three: monoclonal antibodies, small molecule tyrosine kinase inhibitors and inhibitors of mTOR (mammalian target of rapamycin).

Vascular endothelial growth factor (VEGF) which is one of the major growth factors that control angiogenesis has become target of research community in production of anti-angiogenic drugs. Bevacizumab (Lucentis™) is a humanised monoclonal antibody that inhibits VEGF-A. It is primarily used in large doses for treating metastatic colorectal cancer, lung, breast and kidney cancers. Pegaptanib sodium (Macugen™) is another anti-angiogenic agent which is a pegylated anti-VEGF aptamer, a single strand of nucleic acid. It binds specifically to VEGF 165, a protein that plays critical role in angiogenesis. Pegatanib is developed for treating neovascular age-related macular degeneration (AMD) (Ng EW and Adamis AP, 2005).

Other forms of treatments targeting VEGF have also been developed. One of the relevant prior arts in this field of technology is disclosed in International Publication no. WO2009/149205. This prior art discloses a cell therapy for delivering soluble VEGF receptor to eye for treating ophthalmic and cell proliferation disorders. In this prior art, new cells lines that express VEGF receptor has been developed by recombinant technology.

Another prior art relevant in this field is described in European Patent no. EP 1423012 B1. This patent teaches a method for isolating and mobilising mammalian stem cells expressing vascular endothelial growth factor receptor 1 (VEGFR-1) and pharmaceutical use of the receptors. The VEGFR-1 is isolated from stem cells of a post natal mammal for use in treatment of anti-angiogenesis. Further, International Publication no. WO 2005/000895 A2 discloses production of VEGF traps. These are antibodies that binds to VEGF and are expressed using host-vector systems like bacterial, yeast, insect, mammalian cell. The expressed VEGF trap protein is then purified and administered to patients having AMD.

However, these treatments use anti-angiogenic agents which have certain limitations. One of such limitation is high frequency of injection to patients. The anti-angiogenic agents do not last long in human body due to natural protein degradation, requiring frequent injection of the drugs to patient. Moreover, frequent injection also leads to increases in cost of treatment of a disease. Also, the anti-angiogenic drugs have negative side effects on patient including bleeding, high blood pressure, breathing problems and numbness. These effects decrease quality of life of the patients.

All of the above prior arts only provide a short-term treatment for angiogenesis as frequently repeated injections of the anti-angiogenesis drugs are needed. Thus, there is a need in the field for an anti-angiogenesis treatment that will last longer and improve quality of life of a patient by reducing the side effects from the treatment.

### SUMMARY OF INVENTION

The present invention consists of several novel features and a combination of parts hereinafter fully described and illustrated in the accompanying description and drawings, it being understood that various changes in the details may be made without departing from the scope of the invention or sacrificing any of the advantages of the present invention.

The present invention provides a genetically-engineered mesenchymal stem cells (MSCs) having a recombinant vector carrying a vascular endothelial growth factor receptor (VEGFR) gene and expressing a vascular endothelial growth factor receptor (VEGFR) polypeptide. The stem cells according to the present invention inhibit angiogenesis in human body especially in a patient suffering from macular degeneration, cancer, diabetic retinopathy, lymphangiogenesis, retinal neovascularisation, thyroid hyperplasia, preeclampsia, rheumatoid arthritis and osteo-arthritis, Alzheimer's disease, obesity, pleural effusion, atherosclerosis, endometriosis, corneal vascularization and choroidal neovascularization.

### BRIEF DESCRIPTION OF THE DRAWINGS

The invention and its various embodiments are better understood by reading the description along with the accompanying drawings which appear herein for purpose of illustration only and does not limit the invention in any way, wherein:

FIG. 1 is an agarose gel electrophoresis photograph of RT-PCR products from passage 3 to 8.

FIG. 2 is a Western blot analysis of WJ-MSC transfected with sFLT-1 plasmids from passage 3 to 8.

FIG. 3 is microscopic images of wound healing of human umbilical vein endothelial cells (HUVEC). Figure (a) represents untreated control at 0 hour; (b) HUVEC cells treated with sFLT-1 at 0 hour; (c) HUVEC cells treated with bevacizumab (0.5 mg/mL) at 0 hour; (d) Untreated control at 48 hours; (e) HUVEC cells treated with sFLT-1 (˜2 ng/mL) at 48 hours, and; (f) HUVEC cells treated with bevacizumab (0.5 mg/mL) at 48 hours.

### DETAILED DESCRIPTION OF THE PREFERRED EMBODIMENT

The present invention relates to inhibition of angiogenesis particularly, in macular degeneration and cancer, by expressing vascular endothelial growth factor receptors (VEGFRs) in genetically-engineered mesenchymal stem cells (MSCs). The MSCs are isolated from Wharton's jelly of umbilical cord. Hereinafter, this specification will describe the present invention according to the preferred embodiment of the present invention. However, it is to be understood that limiting the description to the preferred embodiment of the invention is merely to facilitate discussion of the present invention and it is envisioned that those skilled in the art may devise various modifications and equivalents without departing from the scope of the appended claims.

The term “mesenchymal stem cells (MSCs)” herein described as multipotent stromal cells having the ability to differentiate into variety of other cells. These stem cells can be derived from bone marrow, umbilical cord, cord blood, peripheral blood, fallopian tube, and fetal liver and lung. Due to the multipotency and low immunogenicity, MSCs are suitable as carriers for therapeutic agents. In particular, the present invention is relevant to the use of mesenchymal stem cells isolated form Wharton's jelly of umbilical cord (WJ-MSCs).

The term “angiogenesis” or “neovascularisation” herein is defined as a physiological process of formation of new blood vessels from pre-existing blood vessels. The growth of new blood vessels is driven by endothelial cell proliferation and migration triggered by pro-angiogenic factors. Angiogenesis facilitates wound healing, growth of hair and fat tissue, nerve regeneration, and muscle and bone repair. However, abnormal formation of blood vessels has harmful effects such as growth of tumours and metastasis, and hemangioma.

The term “anti-angiogenic agent” or “anti-angiogenic protein” herein describes compounds that disrupt angiogenesis. Angiogenesis requires binding of signaling molecules, such as vascular endothelial growth factors (VEGFs), to its receptors on surface of normal endothelial cells to promote growth and survival of new blood vessels. In this invention, the term “anti-angiogenic agent” refers to VEGFRs, in particular VEGFR-1 and VEGFR-2.

The term “VEGFR polypeptide” herein describes a polypeptide that can bind to VEGF in order to render it non-functional. The VEGFR polypeptide defined in this invention is any polypeptide having 50 to 99% homology in protein sequence to sequence of SEQ ID No.1.

The term “inhibiting angiogenesis” used herein means reduction or prevention of formation of new blood vessels. Inhibition of angiogenesis includes slowing the rate of new blood vessel formation. Inhibition in this context also means no further formation of new blood vessels upon administering the anti-angiogenic agent.

The term “angiogenic disease or disorder” and “angiogenesis-related disease” as used in this invention refers to particularly to any disease or disorder caused by uncontrolled or increased growth of new blood vessels. The diseases or disorders can be as a direct result of abnormal blood vessel proliferation. The term also refers to diseases or disorders with pathological progression that requires blood supply and therefore, blood vessel proliferations. Examples of such disease and disorders include but are not limited to abnormal vascular proliferation, macular degeneration, cancer, diabetic retinopathy, lymphangiogenesis, retinal neovascularisation, thyroid hyperplasia, preeclampsia, rheumatoid arthritis, corneal vascularisation, osteo-arthritis, Alzheimer's disease, obesity, pleural effusion, atherosclerosis, endometriosis and choroidal neovascularization.

The term “therapeutically effective amount” or “effective amount” used herein refers to a sufficient amount of composition administered to a patient suffering from the angiogenic disease or disorder to cure or at least partially arrest the disease or disorder.

### Genetically-Engineered WJ-MSCs and a Method of Production Thereof

Mesenchymal stem cells (MSCs) are derived from post-natal or adult tissues, such as umbilical cord, bone marrow, adipose tissues or muscle. Due to nature of source, MSCs are more acceptable for treatment of diseases than embryonic stem cells as MSCs do not pose ethical problems.

The present invention particularly uses the MSCs isolated from the Wharton's jelly of umbilical cord known as WJ-MSCs. WJ-MSCs are multipotent and are non-invasiveness in procurement. WJ-MSCs are easily resourced and have low immunogenicity. Further, WJ-MSCs have been reported to actively migrate and home to sites of injury, inflammation and tumour, making these stem cells uniquely suited as carrier for therapeutic agents.

In the present invention, genetically-engineered WJ-MSCs act as carriers for anti-angiogenic proteins are produced. The WJ-MSCs expressing anti-angiogenic proteins are used to reduce or inhibit angiogenesis in conditions like diabetic retinopathy, age-related macular degeneration or cancer.

The anti-angiogenic protein used in the present invention is a vascular endothelial growth factor receptor (VEGFR). This receptor can be classified into three types: VEGFR-1, VEGFR-2 and VEGFR-3. In the present invention, the genetically-engineered stem cells express VEGFR-1, VEGFR-2 or a combination of both. Vascular endothelial growth factor receptor-1 (VEGFR-1), also known as fms-related tyrosine kinase 1 (FLT-1) in human, is a receptor tyrosine kinase (RTK) specific for the angiogenic factors VEGF such as VEGF-A, VEGF-B and placental growth factor (PIGF). VEGFR-1 is expressed in two forms via alternate splicing at the pre-mRNA level: a full-length, membrane bound receptor capable of transducing signal and a truncated, soluble receptor (sVEGFR-1) capable of sequestering ligand or dimerizing with full-length receptor and preventing signal transduction.

Human VEGFR-1 gene produces two major transcripts of 3.0 and 2.4 kb, corresponding to the full-length receptor and soluble receptor, respectively. Full length VEGFR-1 is an approximately 180 kDa glycoprotein featuring seven extracellular immunoglobulin (Ig)-like domains, a membrane spanning region, and an intracellular tyrosine kinase domain containing a kinase insert sequence. The truncated sVEGFR-1 consists of only first six extracellular Ig-like domains. Ligand binding takes place within the first three N-terminal Ig-like domains while the fourth Ig-like domain is responsible for receptor dimerization, which is a prerequisite for activation through transphosphorylation. In addition to homodimers, VEGFR-1 can form active heterodimers with VEGFR-2. Soluble form of VEGFR-1 forms inactive heterodimers with VEGFR-2. VEGFR-2 is known as KDR (kinase insert domain receptor) in humans or FLK-1 (fetal liver kinase-1) in mice. Like VEGFR-1, VEGFR-2 also contains seven Ig-like repeats within its extracellular domains and kinase insert domains in its intracellular regions.

These receptors play essential roles in angiogenesis. VEGFR-2 binds VEGF-A (VEGF121, VEGF165, VEGF189 and VEGF206 splice variants), VEGF-C and VEGF-D. Full-length cDNA for VEGFR-2 encodes a 1356 amino acid (aa) precursor protein with a 19 aa signal peptide. The mature protein is composed of a 745 aa extracellular domain, a 25 aa transmembrane domain and a 567 aa cytoplasmic domain.

In contrast to VEGFR-1, which binds both PIGF and VEGF with high affinity, VEGFR-2 binds VEGF with high affinity but not PIGF. Soluble forms of VEGFR-1 and VEGFR-2 also differ significantly from one another in terms of their abilities to block VEGF-induced cell proliferation and migration. Soluble VEGFR-2 cannot compete with soluble VEGFR-1 for binding with VEGF in human endothelial cells expressing both VEGFR-1 and VEGFR-2. This is because soluble VEGFR-2 can only partially inhibit cell migration, whereas soluble VEGFR-1 can almost completely block VEGF-induced cell proliferation and migration (Roeckl W. et al., 1998).

The present invention discloses WJ-MSCs expressing anti-angiogenic proteins VEGFR-1 and VEGFR-2. The expressed proteins can be in their full-length or in a truncated form. If the proteins are in their truncated form, the binding site of these proteins to VEGF must be functional. Preferably, the anti-angiogenic proteins are in soluble form and more preferably, anti-angiogenic protein is a soluble form of FLT-1. The expressed protein has a sequence as described in SEQ ID No.1, protein sequence of FLT-1 (UniProt identifier: P17948-1). In another embodiment of the invention, the protein has sequence that is about 50% to 99% homologous to SEQ ID No.1.

Further, the present invention teaches a method for producing genetically-engineered MSCs that express anti-angiogenic proteins. The said genetically-engineered MSCs are produced using recombinant technology. The stem cells are the host cells that express desired protein and having a recombinant vector carrying gene encoding the desired protein. The stem cells are preferably mesenchymal stem cells sourced from Wharton's jelly of umbilical cord.

A recombinant vector having gene for expressing the anti-angiogenic protein is used to transfect the stem cells. The recombinant vector is a plasmid vector or a viral vector.

Preferably, the vector used in this invention is a plasmid vector and more preferably, the plasmid vector is pBLAST-hsFLT-1, marketed by Invivogen®. This plasmid carries the genes for soluble form of FLT-1 protein, expressing the proteins with the sequence of SEQ ID No.1.

According to the present invention, the WJ-MSCs are transfected with pBLAST-hsFLT-1 vector using a suitable transfection method known in the art such as electroporation, lipid-mediated delivery, biolistics particle delivery, and virus-mediated delivery. Preferably, the stem cells are transfected by chemical transfection method. As one skilled in the art understands, transfecting stem cells are difficult to do. Protocols and parameters of electroporation needs to adjusted depending on the type of cells used. According to the present invention, the method is cationic lipid transfection method.

Prior to the transfection, the WJ-MSCs are seeded in a culture medium comprising of VascuLife® EnGS Medium (LifeLine, US) to achieve 90-95% confluent at the time of transfection. Ratio of the DNA solution to cationic transfection reagent is 1:2. The DNA solution and the transfection reagent were incubated for 5 hours and at 37° C.

The transfected stem cells are selected using a negative selection antibiotic marker available on the recombinant vector. For pBLAST-hsFLT-1 vector, the antibiotic marker is Blasticidin. The stem cells that are not transfected are found to be killed by Blasticidin with concentration of at least 10 μg/ml. This concentration is used to select the positively transfected WJ-MSCs.

In another embodiment of the present invention, the genetically-engineered stem cells are harvested in a culture medium comprising of VascuLife® EnGS Medium (LifeLine, US).

Then, the VEGFRs expressed by the harvested stem cells are isolated using a protein extraction (i.e cell lysis) and protein purification method known in the art.

### Use of Genetically-Engineered Stem Cells

According to an embodiment of the present invention, the genetically-engineered MSCs expressing an anti-angiogenic protein is used to inhibit angiogenesis in a human body. The inhibition of angiogenesis is desired in patients suffering from a disease or disorder caused directly by abnormal formation of blood vessels such as macular degeneration, lymphangiogenesis and endometriosis. Also, other diseases and disorders that require blood supply for its progression i.e. cancer and metastasis, can also be treated by inhibiting angiogenesis.

In particular, the present invention discloses a use of the WJ-MSCs for inhibiting angiogenesis in order to treat a disease associated with abnormal growth of blood vessels. The said disease includes but not limited to macular degeneration, cancer, diabetic retinopathy, lymphangiogenesis, retinal neovascularisation, thyroid hyperplasia, preeclampsia, rheumatoid arthritis and osteo-arthritis, Alzheimer's disease, obesity, pleural effusion, atherosclerosis, endometriosis, corneal vascularization and choroidal neovascularization. More preferably, the present invention aims to treat macular degeneration, cancer and diabetic retinopathy.

The genetically-engineered MSCs used in the treatment expresses an anti-angiogenic protein, preferably, a VEGFR. In particular, the VEGFR is a soluble human FLT-1. The VEGFR binds to VEGF to inhibit endothelial cell proliferation and vascular permeability. According to an embodiment of the present invention, the VEGFR is VEGFR-1. VEGFR-2 can be expressed following the method described in the present invention. It is also possible to administer both types of receptors to a patient in order to inhibit angiogenesis.

The MSCs used in the treatment are administered to a patient by injection into veins or by direct injection to an affected part of the patient. The MSCs administered to the patient is a pharmaceutically effective amount. The pharmaceutically effective amount is to be determined by a certified physician treating a patient and can be increased as per the physician's prescription.

Upon the first administration, MSCs in the patient body may last for approximately 1.5 to 2 months, depending on the patient's health condition and the progression of the disease. After this period of time, the count of MSCs may fall beyond pharmaceutically effective amount. Therefore, the MSCs need to be re-injected into the patient after 1.5 to 2 months from the first injection or when the stem cells are depleting and the cell count is below pharmaceutically effective amount.

In another embodiment of the present invention, the expressed VEGFRs are isolated from the MSCs and are used for a treatment involving inhibition of angiogenesis. The isolated VEGFRs are used to administer to a patient via injection into veins or via injection to affected body part.

### Pharmaceutical Compositions a Kit Thereof

In one embodiment of the present invention, a pharmaceutical composition for inhibiting angiogenesis is provided. The pharmaceutical composition comprises of genetically-engineered stem cells expressing VEGFR and a pharmaceutically acceptable carrier. The said pharmaceutical composition is used in treatment of a disease that requires inhibition of angiogenesis, particularly in treatment of macular degeneration, cancer and diabetic retinopathy.

The term “pharmaceutically acceptable carriers” as used herein include diluent, adjuvant, excipients, stabilizers, vehicle or support which are nontoxic to the cell or mammal being exposed thereto at the dosages and concentrations employed. Often the carrier is an aqueous pH buffered solution, antioxidants, low molecular weight (less than about 10 residues) polypeptide, hydrophilic polymers, amino acids such monosaccharides, disaccharides, chelating agents such as EDTA, salt-forming counterions such as sodium; and non-ionic surfactants such as TWEEN®, polyethylene glycol (PEG), and PLURONICS® In particular, for compositions administered intravenously, a saline solution is the preferred carrier.

The pharmaceutical composition of the invention may be in a variety of forms. These include, for example, liquid dosage forms, such as lyophilized preparations, liquid solutions or suspensions, injectable and infusible solutions, etc. The pharmaceutical composition is preferably injectable. The pharmaceutical compositions of the invention may also be combined with other types of treatments like steroid, non-steroidal, anti-angiogenesis compounds, or other agents useful in inhibiting angiogenesis.

A kit comprising a container and a composition contained therein, wherein the composition comprises a genetically-engineered mesenchymal stem cells in a culture medium expressing VEGFR. According to the present invention, the culture medium is VascuLife® EnGS Medium (LifeLine, US). The said kit further optionally comprises a package insert indicating the composition can be used to inhibit angiogenesis. The composition in the kit is used to treat macular degeneration, cancer and diabetic retinopathy.

An embodiment of the present invention is described herein.

### Example

### Primer Design

The primers used in this study were designed based on the sFLT-1 and Glyceraldehyde 3-phosphate dehydrogenase (GAPDH) genes. The GAPDH gene is used as control (housekeeping gene), of which the expression remains constant in cells. The primers were designed using FastPCR 4.0.13 software (Institute of Biotechnology, University of Helsinki, Finland). All primers were synthesized by First Base Laboratories, Malaysia. The primer sequences are listed in Table 1.

TABLE 1
List of primers used in amplification and
detection of FLT-1 and GAPDH transcripts.

| | | Expected gene size |
|---|---|---|
| Primer Name | Primer Sequence | (bp) |
| sFLT-1 Forward | 5′ CCA TCA GCA GTT CCA CCA CT 3′ | FLT-1 gene (204) |
| sFLT-1 Reverse | 5′ ACA CAG AGC CCT TCT GGT TG 3′ | |
| GAPDH Forward | 5′ GACCACAGTCCATGCCATCA 3′ | GAPDH gene (453) |
| GAPDH Reverse | 5′ TCCACCACCCTGTTGCTGTA 3′ | |

Overview of pBLAST-hsFLT-1 Vector

pBLAST-hsFLT-1 (manufactured by Invivogen®) expressing a soluble form of human FLT-1 (VEGFR-1) ORF. pBLAST is a ready-made expression vector containing a gene of interest from the angiostatic, angiogenic, growth factor, or differentiation inhibitor family that will produce angiostatic and angiogenic proteins in vitro and in vivo.

### Recovery of Frozen Stock WJ-MSCs

The WJ-MSCs were removed from liquid nitrogen and thawed by continuously swirling in a 37° C. water bath until a slight amount of ice remains. The vial was cleaned with alcohol before the cap was opened. 1 mL of cells suspension was transferred to a 25 cm² T-flask containing 5 mL of VascuLife® EnGS Medium (LifeLine, US). F12 medium (Gibco, USA) supplemented with 10% (v/v) of Fetal Bovine Serum (FBS) (Gibco, USA). The flask was incubated at 37° C. and the next day, the medium was removed and fresh medium was added. For subculturing, the medium was removed from the flask and rinsed with 1× PBS (137 mM NaCl, 2.7 mM KCl, 4.3 mM Na₂HPO₄, 1.47 mM KH₂PO₄, pH 7.4) and gently rock the flask back and forth. One mL of the pre-warmed TrypLE® (Gibco, USA) was added to the flask and incubated at 37° C. for 5 min. The flask was gently tapped to dislodge remaining cells that adhered to the flask surface. 5 mL of complete growth media was added and cells were re-suspended by pipette repeatedly to break up any clumps that may be present. The cells were counted using hemocytometer and the cells were spilt at ratio of 1:3 to new flasks for subculturing. Cell concentration was determined by the formula (Liddell and Cryer, 1991).

$$\frac{Mean of counted cells}{1 ml} \times {10}^{4} \times dilution factor = x cells \text{/} ml$$

### Cryopreservation of WJ-MSCs

The WJ-MSCs were stored during log phase where >90% of the cells were viable. Cells at the concentration of 5×10⁵ cells per mL were centrifuged at 200×g for 10 min. Pellet from the centrifugation was re-suspended gently in 2 mL of chilled freezing medium containing 20% (v/v) FBS and 10% (v/v) dimethysulphoxide (DMSO). The cells were aliquot in cryovials and placed in styroform box. The cells were kept at −20° C. for 1 h and transferred to −80° C. for overnight prior being transferred to liquid nitrogen for long term storage.

### Determine the Concentration of Blasticidin

The toxicity concentration of Blasticidin (Invivogen, USA) to the WJ-MSCs was determined prior to stable transfection. WJ-MSCs were seeded in 6-well plate (Nunc, USA) at density of 1×10⁵ cells/mL and grown to 90% confluent. Blasticidin concentration with a range between 2 μg/mL and 10 μg/mL each was added to the WJ-MSCs and incubated for 5 days. The cytotoxic effect was determined by evaluating the percentage of cell confluency. It was found that the lowest concentration of Blasticidin that completely inhibits the growth of WJ-MSCs is 10 μg/mL.

Transfection of WJ-MSCs with Human FLT-1 Gene

The WJ-MSCs were transformed using cationic lipid transfection. The WJ-MSCs were seeded in 6-well plate with VascuLife® EnGS Medium (LifeLine, US) without antibiotic a day before the transfection to achieve 90-95% confluent at the time of transfection. 3 μg of purified plasmid DNA was diluted into 100 μL of OPTI-MEM I reduced-serum medium (Gibco, USA) to prepare DNA solution and 6 μL of Lipofectamine® (Invitrogen, USA) reagent was diluted into 100 μL of OPTI-MEM® I reduced-serum medium to prepare the lipid solution. The diluted DNA was mixed gently with the diluted lipid reagent followed by incubation of both solutions for 30 min at RT. Both solutions were then mixed together and incubated for 15 min at RT to allow DNA-Lipid complexes to form. While waiting for the complexes to form, the WJ-MSCs were rinsed with pre-warmed VascuLife® EnGS Medium (LifeLine, US) medium without serum as serum could lower the Lipofectamine® reagent performance. 2 mL of OPTI-MEM® I medium was added to the DNA-Lipid complexes and mixed gently. The rinsed cells were overlaid slowly with the DNA-Lipid complexes. After 5 hours of incubation at 37° C., 2 mL of VascuLife® EnGS medium (LifeLine, US) containing 20% serum was added onto the cells without removing the transfection mixture. Next day, the cells medium was removed and added with VascuLife® EnGS Medium (LifeLine, US) with 10% FBS serum. Transfection of WJ-MSC with FLT-1 gene results in higher efficiency with the combinations of DNA solution to Lipofectamin® at the ratio of 3 μg to 6 μg.

Extraction of Total RNA from WJ-MSCs

RNA extraction was carried out by mixing approximately 400 μL of the WJ-MSCs cells homogenate with 750 μL TRIzol reagent (Gibco, USA) in a 1.5 mL Eppendorf Tube® and incubated for 5 min at room temperature (RT). A 200 μL of chloroform was added and mixed for 15 s. It was incubated at RT for 15 min. The mixture was then centrifuged at 12000×g for 20 min at 4° C. The aqueous phase was transferred into a new 1.5 mL Eppendorf Tube® and RNA was precipitated by adding 800 μL of isopropanol. After 10 min of incubation at RT, the mixture was centrifuged at 12000×g for 15 min at 4° C. The supernatant was removed and the resulting pellet was washed by adding 1 mL of 100% ethanol and centrifuged at 12000×g for 15 min at 4° C. The supernatant was removed again and added with 1 mL of 100% ethanol and kept at −70° C. for further use or centrifuged at 12000×g for 5 min at 4° C., air-dried and suspended in 20 μL RNAaes free water. The extracted RNA was treated with 2 μL DNase I (Sigma, UK) at 37° C. for 30 min. The reaction was stopped with 2 μL of 50 mM EDTA and heat inactivation at 56° C. for 10 min and the RNA was subjected for further analysis.

### Reverse Trascription Polymerase Chain Reaction (RT-PCR) Analysis

RT-PCR analysis was performed on WJ-MSCs to determine the presence of mRNA transcripts. This was done by using the primers as listed in Table 1. A total of 25 μL RT-PCR mixture containing 5 μL AMV/Tfl 1× Reaction Buffer (Promega, USA), 0.5 μL of 0.2 mM dNTP mixture (Promega, USA), 3 μL of 3 mM MgSO4, 0.5 μL of 0.5 μM of each primer (Vivantis, Malaysia), 0.5 μL of 0.8 u/μL RNasin® Ribonuclease Inhibitor (Promega, USA), 0.5 μL of 0.1 u/μL AMV Reverse Transcriptase (Promega, USA), 0.5 μL of 0.1 U/μL Tfl DNA Polymerase (Promega, USA), 1 μL of RNA (10 μg/μL) and 13 μL of Nuclease-Free water. The assay was optimized in respect of annealing temperature, concentration of MgSO₄ and cycling parameters. The assay was carried out in duplicate to demonstrate reproducibility. The mixture was mixed properly by vortex and centrifugation on microcentrifuge at RT. Gradient PCR was performed for the first run at 45° C. for 45 min for one cycle as reverse transcription and by 34 cycles of pre-denaturation step at 95° C. for 2 min, denaturation at 95° C. for 30 s, annealing at 50-65° C. for 44 s, extension at 68° C. for 2 min, followed by the final extension at 68° C. for 10 min in Eppendorf® Thermal Cycler PCR system (Eppendorf, USA). An annealing temperature of 55° C. was evaluated to give maximum product yields and specificity for all the primer sets. The RT-PCR products were run on agarose gel and subjected to electrophoresis at 80V for 50 min. The gel was stained with GelRed™ (Biotium, USA) and visualized under BioSpectrum® (UVP, USA).

### SDS-Polyacrylamide Gels Electrophoresis (SDS-PAGE)

One 12% resolving gel was prepared from 940 μL of 30% monomer solution [29.2% w/v) acrylamide, 0.8% (w/v) bisacrylamide], 2.5 mL of 920 μL Tris (pH 8.8), 20 μL of 10% (w/v) SDS, 940 μL of dH2O, 23.5 μL of 10% (w/v) ammonium persulfate (APS) and 3.8 μL of N,N,N′,N′-tetramethylethylenediamine (TEMED). All the components were mixed and pipetted in between two casting glass plates. The gel was overlaid with 0.1 mL of 100% butanol and allowed to polymerise for approximately 15 min. Then butanol was discarded and rinsed with distilled water (dH₂O). Stacking gel solution [415 μL of 30% monomer solution, 588.8 μL of 0.5 M Tris-Cl (pH 6.8), 36.2 μL of 10% (w/v) SDS, 1.46 mL of dH2O, 16.7 μL of 10% (w/v) APS and 3.5 μL of TEMED] was layered on top of the resolving gel. The combs were inserted into the stacking gels and left to polymerise for about 30 min. Reservoir tank was filled with electrophoresis buffer [25 mMTris, 250 mM glycine, 0.1% SDS, pH 8.3]. The assay was carried out in duplicate to demonstrate reproducibility. The samples were mixed with equal volume of 2× sample buffer [0.5 M Tris (pH 6.8), 100% glycerol, 10% (w/v) SDS, 0.5% (w/v) bromophenol blue, 10% (v/v) β-mercaptoethanol] and short spun before and after heating at 100° C. for 10 min. Electrophoresis apparatus was set at constant current of 16 mA until the sample buffer ran off. The gels were then stained in staining solution [0.025% (w/v) Coomassi® brilliant blue R-250, 40% (v/v) methanol, 7% (v/v) acetic acid] for 30 min followed by destaining in destaining solution [40% (v/v) methanol, 7% (v/v) acetic acid] until the background stain was clear. The sample sizes were measured corresponding to the MagicMark™ XP Western Protein Standard (Invitrogen, USA).

### Western Blot

SDS-PAGE with protein samples were subjected to electro-transfer without prior staining. Polyacrylamide gel containing the electrophoresed samples was arranged into sandwich position in the following steps; first, three layers of 3 mm chromatography papers (Whatman, USA) followed by nitrocellulose membrane (GE healthcare, USA), then polyacrylamide gel and finally another three layers of chromatography papers. All layers were previously soaked in Towbin's transfer buffer [25 mMTris, 190 mM glycine, 20% (v/v) methanol, pH 8.0] (Towbin et al., 1979) before arranging onto the Trans-blot SD semi-dry electrophoretic transfer cell (BioRad, USA). Samples were blotted to membrane with a constant voltage of 15 V for 15 min. The membrane was incubated in 7 mL of primary antibodies against sFLT-1 (Abcam, USA) diluted at 1:1000 and incubated at RT for 1 h to detect the sFLT-1 protein. The membrane was washed with 10 mL of 1×TBST (2.5 g milk in 50 ml 1×TBST) and the process was repeated thrice. The membrane was then incubated in 7 mL of anti-rabbit secondary antibodies conjugated with HRP (Abcam, USA) for 1 h. Again the membrane was covered with 10 mL of 1×TBST and repeated thrice. Subsequently, the blot was incubated with 5 mL of ECL mix (GE Healthcare, USA) and immediately exposed to chemiluminescence for 10-30 mins.

### In Vitro Scratch Assay

In order to further understand the anti-angiogenesis activity of sFLT-1, a wound-healing assay was conducted in accordance to Liang et al. (2007). This assay was attempted in human umbilical vein endothelial cells (HUVEC) treated with sFLT-1 in comparison with HUVEC without treatment and HUVEC treated with 0.5 mg/mL. Bevacizumab is a monoclonal antibody that inhibits VEGF-A. This assay is done to visualise the cell migration in light of suppression of FLT-1 gene. In vitro scratch assay mimics to some extent migration of cells in vivo.

### Analysis

### Stable Transfection of WJ-MSCs

The effect of concentration of Blasticidin against the WJ-MSCs at day-1 to day-5 was analysed. The least viable cells were observed when WJ-MSCs were treated with 10 μg/mL of Blasticidin which cause rounding and floating of WJ-MSCs indicated cells death occurs. The lowest concentration of Blasticidin that completely inhibited the growth of WJ-MSCs was found to be 10 μg/mL. This concentration was used in selection of the positive transfected WJ-MSCs, and Blasticidin concentration of 4 μg/mL was used for maintenance of WJ-MSCs after transfection with DNA constructs.

Detection of mRNA Transcript in Transfected Cells

The transfected WJ-MSCs were analysed for the presence of sFLT-1 mRNA transcript. The extracted total RNA was subjected to RT-PCR using the primers listed in Table 1. The RT-PCR analysis revealed that the plasmids were transcriptionally active in transfected WJ-MSCs. In order to ensure the amplification results was associated with the transcripts and not from the plasmid DNA, the extracted RNA samples were processed for PCR amplification without the RT part. No specific band was detected from the samples and this indicates that the amplification result was not from plasmid DNA.

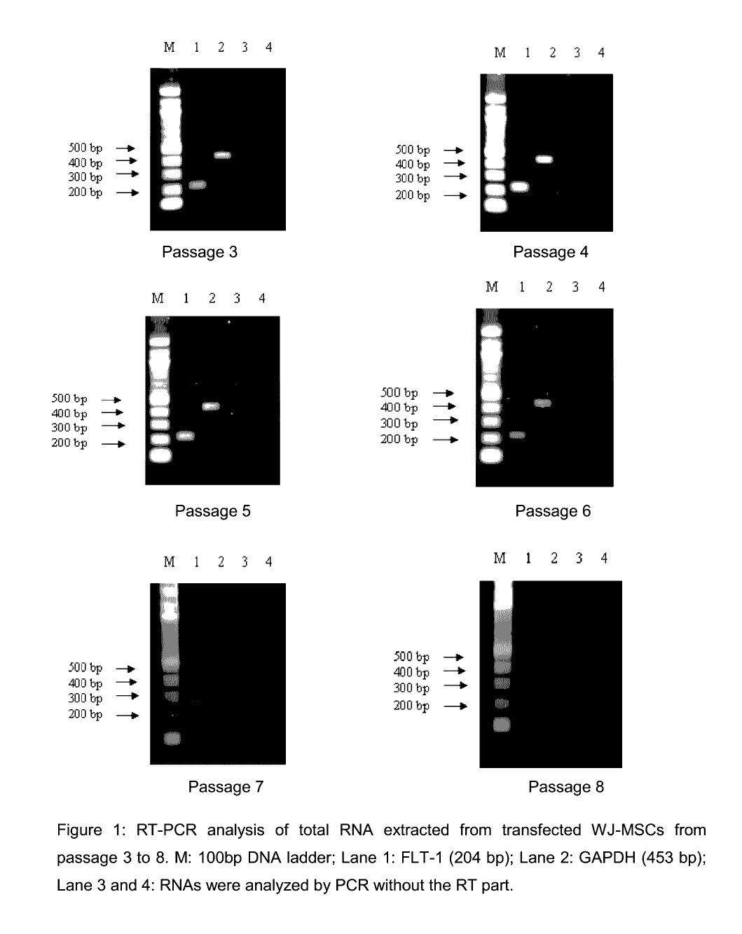

The RT-PCR analysis revealed that the plasmids were transcriptionally active in transfected WJ-MSCs (FIG. 1).

Transfection Efficiency of pMAX-GFP in WJ-MSCs

To aid in the in vivo tracking of WJ-MSCs expressing FLT-1, cells were transfected with pMAX-GFP using nucleofection method. Although little positive clones were obtained (in part due to low transfection efficiency and the lack of selective pressure), a small population of WJ-MSCs continued to express green fluorescent protein (GFP) up to passage 5.

### Western Blot Analysis

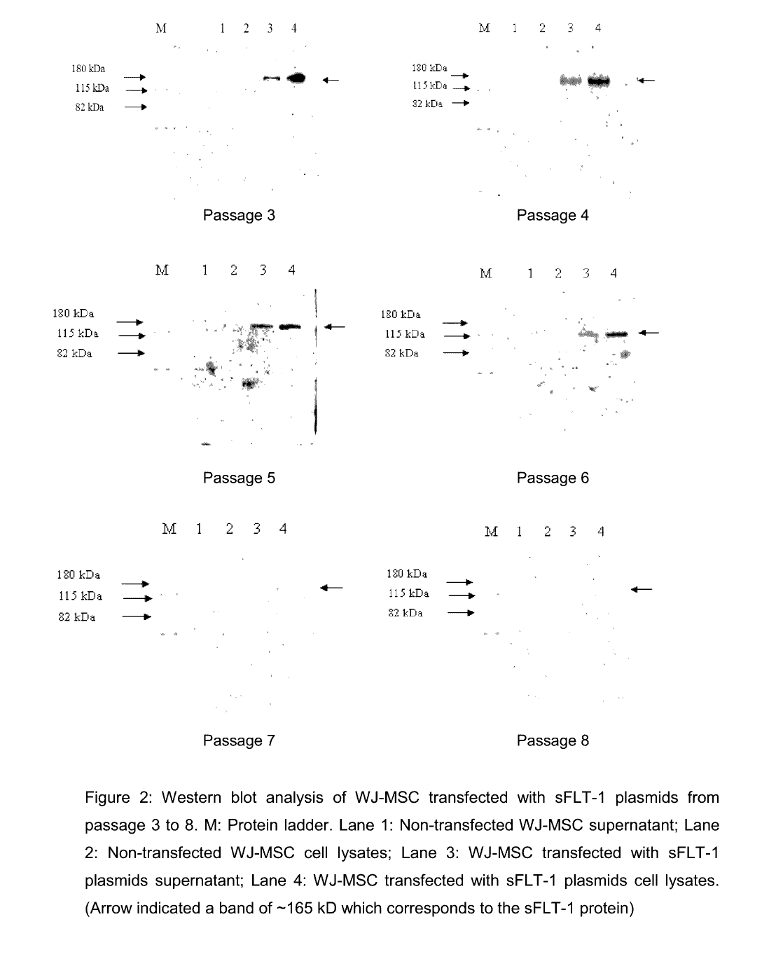

After 2 weeks of Blasticidin treatment on WJ-MSCs, cell lysates were collected and separated by SDS-PAGE. It was followed by electro-transfer of the SDS-PAGE protein samples onto the nitrocellulose membrane and probed with primary and secondary antibodies. The expressions of FLT-1 proteins were confirmed by Western blot analysis. Molecular weight species of approximately 165 kDa in pBLAST-hsFLT-1 transfected cells reacted with primary antibodies against sFLT-1 and anti-rabbit secondary antibodies conjugated with HRP (FIG. 2). The FLT-1 protein was highly expressed in passage 3 and the protein level was decreasing in Passage 8. It took 1 week for each passage until passage 8.

The present invention also showed transfection of pBLAST-hsFLT-1 plasmids into WJ-MSCs was able to induced expression of FLT-1 protein which last up to passage 8 or 2 months. Compared to administration of other anti-angiogenic agents, the genetically-engineered WJ-MSCs expressing sFLT-1 last longer in patient and do not require frequent injections.

### In Vitro Scratch Assay

The migration of cells towards the centre of the wound for HUVEC cells treated with sFLT-1 was faster compare to HUVEC treated with 0.5 mg/mL bevacizumab. In conclusion, partial inhibition was achieved for HUVEC treated with sFLT-1 compare to 0.5 mg/mL bevacizumab.

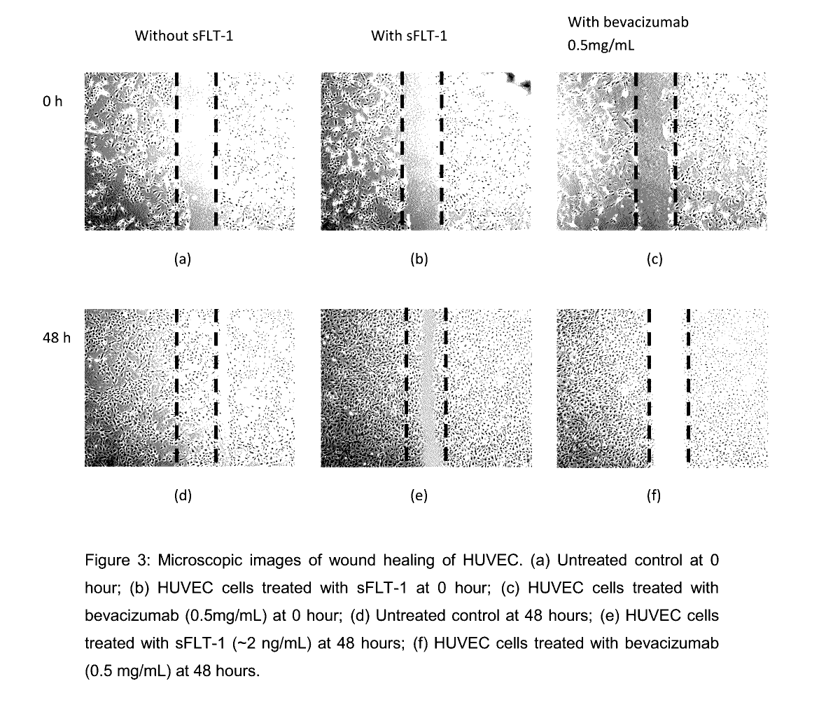

FIG. 3 shows the results of the assay at 0 hour and 48 hours. From figure (c) and (f), it is clear that treatment with bevacizumab (0.5 mg/mL) significantly inhibited HUVEC migration exemplifying role of bevacizumab in inhibition of cell migration. Similar effects were found with the experiment replicated with sFLT-1 treatment. Figure (e) shows that the HUVEC migration was partially inhibited with sFLT-1 treatment. In the control, figure (a) and (d), the HUVEC migration was not inhibited in absence of any treatments.

Result of this assay demonstrates cells expressing FLT-1 proteins to inhibit angiogenesis in vitro and also the feasibility of the same in vivo. It shows the potential of the genetically-engineered cells to reduce cell migration activities and in turn reduce the occurrence of angiogenesis in a patient.

SEQUENCE LISTING

```
SEQ ID. No.1
                10 20 30 40
MVSYWDTGVL LCALLSCLIL TGSSSGSKLK DPELSLKGTQ
                50 60 70 80
HIMQAGQTLH LQCRGEAAHK WSLPEMVSKE SERLSITKSA
                90 100 110 120
CGRNGKQFCS TLTLNTAQAN HTGFYSCKYL AVPTSKKKET
              130 140 150 160
ESAIYIFISD TGRPFVEMYS EIPEIIHMTE GRELVIPCRV
              170 130 190 900
TSPNITVTLK KFPLDTLIPD GKRIIWDSRK GFIISNATYK
              210 290 230 240
EIGLLTCEAT VNGHLYKTNY LTHRQTNTII DVQISTPRPV
              250 260 270 230
KLLRGHTLVL NCTATTPLNT RVQMTWSYPD EKNKRASVRR
              990 300 310 320
RIDQSNSHAN IFYSVLTIDK MQNKDKGLYT CRVRSGPSFK
              330 340 350 360
SVNTSVHTYD KAFITVKHRK QQVLETVAGK RSYRISMKVK
              370 380 390 400
AFPSPEVVWL KDGLPATEKS ARYLTRGYSL IIKDVTEEDA
              410 420 430 440
GNYTILLSIK QSNVFKNITA TLIVNVKFQ1 YEKAVSSFPD
              450 460 470 480
PALYPLGSRQ ILTCTAYGIP QPTIKWFWHP CNHNHSEARC
              490 500 510 520
DFCSNNEESF ILDADSNMGN RIESITQRMA IIEGKNKMAS
              530 540 550 560
TLVVADSRIS GIYICIASNK VGTVGRNISF YITDVPNGFH
              570 580 590 600
VNLEKMPTEG EDLKLSCTVN KFLYRDVTWI LLRTVNNRTM
              610 620 630 640
HYSISKQKMA TTKEHSTTMN LTIMNVSLQD SGTYACRARN
              650 660 670 680
VYTGEEILQK KEITIRDQEA PYLLRNISDH TVAISSSTTL
              690 700 710 720
DCHANGVPEP QITWFKNNHK TQQFPGITLG PGSSTLFIER
              730 740 750 760
VTEEDEGVYH CEATNQKGSV ESSAYLTVQG TSDKSNLELI
              770 780 790 800
TITCTCVAAT LEWLLLTIFI REMKRSSSEI KTDYLSIIMD
              810 820 830 840
PDEVPLDEQC ERLPYDASKW EFARERLKLG KSLGRGAFGK
              850 860 870 830
VVQASAFGIK KSPTCRTVAV KMLKEGATAS EYNALMTELN
              890 900 910 920
ILTHIGHHLN VVNLLGACTK QGGPLMVIVE YCKYGNLSNY
              930 940 950 960
LKSKRDLFFL NKDAALHMEP KKEKMEPGLE QGKKPRLDSV
              970 930 990 1000
TSSESFASSG FQEDESLSDV EEEEDSDGFY KEPITMEDLI
            1010 1020 1030 1040
SYSFQVARGM EFLSSRECTH RDLAARNILL SENNVVKICD
            1050 1060 1070 1080
FGLARDIYKN PDYVRKGDIR LPLKWMAPES IFDKIYSTKS
            1090 1100 1110 1120
DVWSYGVLLW EIFSLGGSPY PGVWDEDFC SRLREGMRMR
            1130 1140 1150 1160
APHYSTPEIY QTMLDCMHRD PKFRPRFAEL VEELGDLLQA
            1170 1180 1190 1200
NVQQDGKDYT PINAILTGNS GFTYSTPAFS FDPFKESTSA
            1210 1220 1230 1240
PKENSGSSDD VRYVNAFKFM SLERIKTFEE LLPNATSMFD
            1250 1260 1270 1280
DYQGDSSTLL ASPMLKRFTW TDSKPKASLK IDLRVTSKSK
            1290 1300 1310 1320
ESGLSDVSRP SFCHSSCGHV SEGKRRFTYD HAELERKIAC
            1330
CSPPPDYNSV VLYSIPPI
```

1. Genetically-engineered mesenchymal stem cells (MSCs) having a recombinant vector carrying a vascular endothelial growth factor receptor (VEGFR) gene and expressing a vascular endothelial growth factor receptor (VEGFR) polypeptide, wherein the said stem cells inhibit angiogenesis in human body.

2. The stem cells as claimed in claim 1, wherein the VEGFR gene is VEGFR1.

3. The stem cells as claimed in claim 1, wherein the said VEGFR polypeptide is a soluble form of VEGFR.

4. The stem cells as claimed in claim 1, wherein the VEGFR polypeptide is a human FLT-1 protein.

5. The stem cells as claimed in claim 1, wherein the recombinant vector is a plasmid or a viral vector.

6. The stem cells as claimed in claim 5, wherein the plasmid vector is pBLAST-hsFLT-1.

7. The stem cells as claimed in claim 1, wherein the mesenchymal stem cells are isolated from umbilical cord.

8. The stem cells as claimed in claim 6, wherein the plasmid vector is transfected into the stem cells by cationic lipid transfection.

9. The stem cells as claimed in claim 1, wherein the stem cells express proteins having an amino acid sequence of SEQ ID NO.1.

10. The stem cells as claimed in claim 9, wherein the stem cells express proteins having sequence 50 to 100% homology to SEQ ID NO.1.

11. The stem cells as claimed in claim 1, wherein the angiogenesis is inhibited in patients having disease or disorder selected from a group comprising of macular degeneration, cancer, diabetic retinopathy, lymphangiogenesis, retinal neovascularisation, thyroid hyperplasia, preeclampsia, rheumatoid arthritis and osteo-arthritis, Alzheimer's disease, obesity, pleural effusion, atherosclerosis, endometriosis, corneal vascularization and choroidal neovascularization.

12. A method for producing stem cells genetically-engineered mesenchymal stem cells as claimed in claim 1, comprising the steps of:
i. transfecting mesenchymal stem cells with a DNA construct comprising a gene encoding for vascular endothelial growth factor receptor (VEGFR) protein;
ii. selecting for expression of the said gene in step (i) in the mesenchymal stem cells; and
iii. culturing the stem cells selected in step (ii).

13. The method as claimed in claim 12, wherein transfection method used in step (i) is cationic lipid transfection.

14. The method as claimed in claim 12, wherein the mesenchymal stem cells are cultured to 90-95% confluency.

15. (canceled)

16. Use of a genetically-engineered mesenchymal stem cells (MSCs) expressing vascular endothelial growth factor receptors (VEGFRs) to inhibit angiogenesis in a patient having disease or disorder associated with uncontrolled growth of new blood vessels.

17. The use as claimed in claim 15, wherein the disease or disorder is selected from a group comprising of macular degeneration, cancer, diabetic retinopathy, lymphangiogenesis, retinal neovascularisation, thyroid hyperplasia, preeclampsia, rheumatoid arthritis and osteo-arthritis, Alzheimer's disease, obesity, pleural effusion, atherosclerosis, endometriosis, corneal vascularization and choroidal neovascularization.

18. The use as claimed in claim 15, wherein the VEGFRs is VEGFR-1.

19. (canceled)

20. A composition comprising of genetically-engineered mesenchymal stem cells (MSCs) capable of expressing soluble vascular endothelial growth factor receptors (VEGFRs) and a pharmaceutically acceptable carrier.

21. The composition as claimed in claim 18, wherein the expressed VEGFRs is human FLT-1.

22. The composition as claimed in claim 18, wherein the mesenchymal stem cells are isolated from umbilical cord.

23. The composition as claimed in claim 18, wherein the pharmaceutically acceptable carrier is a saline solution.

24. A kit comprising a container and a composition contained therein, wherein the composition comprises a genetically-engineered stem cells of claim 1.

25.-26. (canceled)


SEQUENCE LISTING (5 sequences)

SEQ ID NO: 1 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 2 - DNA, 20 bp, Artificial Sequence
  Description of Artificial Sequence Synthetic primer
ccatcagcagttccaccact

SEQ ID NO: 3 - DNA, 20 bp, Artificial Sequence
  Description of Artificial Sequence Synthetic primer
acacagagcccttctggttg

SEQ ID NO: 4 - DNA, 20 bp, Artificial Sequence
  Description of Artificial Sequence Synthetic primer
gaccacagtccatgccatca

SEQ ID NO: 5 - DNA, 20 bp, Artificial Sequence
  Description of Artificial Sequence Synthetic primer
tccaccaccctgttgctgta